(19) United States
(12) Patent Application Publication
(10) Pub. No.: US 2008/0167399 A1
(43) Pub. Date: Jul. 10, 2008

(54) COMPOSITIONS FOR DENTAL COMPOSITES WITH TRICYCLO[5.2.1.02.6]DECANE DERIVATIVES

(75) Inventors: Andreas Utterodt, Neu-Anspach (DE); Klaus Ruppert, Maintal (DE); Matthias Schaub, Linsengericht (DE); Christine Diefenbach, Dornburg (DE); Kurt Reischl, Merenberg (DE); Alfred Hohmann, Schmitten (DE); Michael Eck, Schmitten (DE); Nelli Schonhof, Braunfels (DE)

Correspondence Address: NORRIS, MCLAUGHLIN & MARCUS, P.A., 875 THIRD AVE, 18TH FLOOR, NEW YORK, NY, 10022, US

(73) Assignee: HERAEUS KULZER GMBH, Hanau (DE)

(21) Appl. No.: 11/953,120

(22) Filed: Dec. 10, 2007

(30) Foreign Application Priority Data
Dec. 20, 2006 (DE) 10 2006 060 983.2

(51) Int. Cl. A61K 6/08

(52) U.S. Cl. 523/116

(57) ABSTRACT

Compositions for dental composites comprising

monomers, crosslinking agents, fillers, initiators,
A the proportion of crosslinking agent being formed in an amount of more than 50% by acrylate monomers with a TCD urethane structure having the general formula

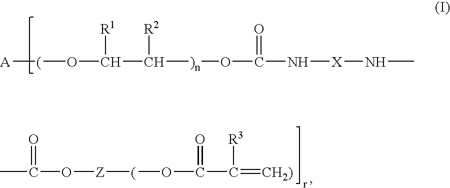

in which A, X, Z, R1 R2 R3 and r have the meaning indicated in claim 1,
provide polymerized composite materials with a particularly low cytotoxicity according to the standard requirements according to ISO 10993-5 and DIN EN ISO 7405.

The invention relates to compositions for dental composites comprising acrylic acid esters of tricyclo[5.2.1.02.6] decane with urethane groups.

### BACKGROUND OF THE INVENTION

Bisphenol A (meth)acrylate monomers have proved to be suitable low shrinkage polymerising monomers for dental filling materials. An alternative to the low shrinkage polymerising bisphenol A (meth)acrylate monomers has been described in EP 0 254 185 (Bayer AG) in the form of TCD monomers. Like the bisphenol A skeleton, the TCD group exhibits the rigidity which causes the low shrinkage polymerisation behaviour. As a result of the steric restriction of the mobility, the urethane derivatives of 1,3-bis(1-isocyanato-1-methylethyl)benzene are very similar in terms of their properties, to bis-GMA and can be used in dental composites in its place, as described in EP 0 934 926.

In concrete terms, however, only the use of the methacrylates is described.

The so-called silorans represent a combination of epoxy functionalities on siloxane units and can be polymerised in a low shrinkage manner via a cationic crosslinking mechanism by ring opening polymerisation. The low shrinkage of these new monomers and the toxicological safety of the otherwise critical epoxides in cured dental composites have been described in DE 100 01 228 and EP 1 117 368.

The higher reactivity of acrylate monomers in comparison with methacrylates is well known; however, the irritant effect vis-à-vis biological tissue is also markedly higher than that of methacrylates, for which reason monomer mixtures with methacrylates, if necessary with small admixtures of acrylates, are mainly used in dental materials. The increased reactivity of urethane (meth)acrylate monomers vis-à-vis polyether monomers, polyester monomers or aliphatic monomers is also well known. Faced with this situation, the task arises of providing dental composites with advantageous properties in spite of the use of acrylate monomers.

### SUMMARY OF THE INVENTION

The invention relates to dental composites comprising monomers, crosslinking agents, fillers, initiators, with the particularities that

A the proportion of crosslinking agent is formed in an amount of more than 50% by acrylate monomers with a TCD urethane structure having the general formula

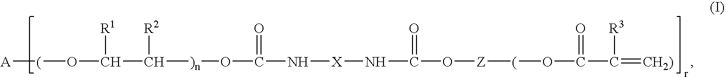

in which
A is a straight-chain or branched aliphatic radical with 2 to 20 carbon atoms, the radical containing, if necessary 1 to 3 oxygen bridges, an aromatic radical with 6 to 24 carbon atoms, an araliphatic radical with 7 to 26 carbon atoms or a cycloaliphatic radical with 6 to 26 carbon atoms,
r represents the number of chains issuing from A and an integer of 2 to 6,
R¹ and R² are identical and represent hydrogen or are different and represent hydrogen and methyl,
n represents, for each chain issuing from A, independently an integer of 0 to 5,
X represents the group

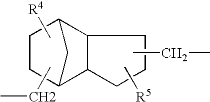

in which
R⁴ and R⁵ are identical or different and represent hydrogen, halogen, lower alkoxy, lower alkyl or trifluoromethyl,
Z may contain a divalent straight-chain or branched aliphatic hydrocarbon radicals of 3 to 15 carbon atoms which, if necessary, may contain 1 to 3 oxygen bridges atoms and, if necessary, may be substituted by 1 to 4 additional (meth)acrylate radicals, R², R³ are exclusively hydrogen and that
B exhibits the cytotoxicity of the hardened composite corresponding to the standard requirements according to ISO 10993-5 and DIN EN ISO 7405, has the assessment “no cytotoxic potential”.

### DETAILED DESCRIPTION OF THE INVENTION

Examples of suitable monomers are

monofunctional or polyfunctional (meth)acrylates, which can be used alone or in mixtures. Examples of such compounds to consider are methylmethacrylate, isobutylmethacrylate, cyclohexylmethacrylate, triethylene glycoldimethacrylate, diethylene glycoldimethacrylate, tetraethylene glycoldimethacrylate, ethylene glycoldimethacrylate, polyethylene glycoldimethacrylate, butandiol dimethacrylate, hexandiol methacrylate, decandiol dimethacrylate, dodecandiol dimethacrylate, bisphenol-A-dimethacrylate, trimethylolpropane trimethacrylate, ethoxylated bisphenol-A-dimethacrylate, but also bis-GMA (2,2-bis-4-(3-methacryloxy-2-hydroxypropyl)phenylpropane) as well as the reaction products from isocyanates, in particular di- and/or triisocyanates and methacrylates that contain OH-groups, and the appropriate acrylates of all the above compounds. Examples of reaction products of isocyanates are the transformation products of 1 mol hexamethylene diisocyanate with 2 mol 2-hydroxyethylmethacrylate, of 1 mol (tri(6-isocyanatohexyl)biuret with 3 mol hydroxy ethylmethacrylate and of 1 mol trimethylhexamethylene diisocyanate with 2 mol hydroxyethylmethacrylate, which are also called urethane dimethacrylates. Suitable monomers are the monomers themselves, polymerizable prepolymers made from them as well as mixtures thereof.

Examples of monomers suitable as crosslinking agents are e.g. 2.2-bis-4-(3-methacryloxy-2-hydroxypropyl)-phenyl propane) (bis-GMA), i.e. the transformation product of glycidyl methacrylate and bisphenol-A (containing OH-groups), and 7,7,9-trimethyl-4,13-dioxo-3,14-dioxa-5,12-diazahexadecan-1,16-diyl-dimethacrylate (UDMA), i.e. the urethane dimethacrylate from 2 mol 2-hydroxyethylmethacrylate (HEMA) and 1 mol 2-2,4-trimethylhexamethylene diisocyanate (containing urethane groups). Furthermore, transformation products of glycidyl methacrylate with other bisphenols, like e.g. bisphenol-B (2,2′-bis-(4-hydroxyphenyl)-butane), bisphenol-F (2,2′-methylene diphenol) or 4,4′-dihydroxydiphenyl, as well as transformation products of 2 mol HEMA or 2-hydroxypropyl(meth)acrylate with, in particular, 1 mol, known diisocyanates, such as e.g. hexamethylene diisocyanate, m-xylylene diisocyanate or toluoylene diisocyanate are preferred as crosslinking monomers. Preferred monomers are bis-GMA, Bisphenol-A-Ethoxydimethacrylate, 2,2-bis[4-(2-hydroxy-3-methacryloxypropoxy)phenyl]propane, polymeric ethoxylated Bisphenol A dimethacrylates (Bis-EMA), Bis EMA (2,6), Bis EMA(6), triethylene glycol dimethacrylate (TEGDMA), 1,6-bis(methacryloxy-2-ethoxycarbonylamino)-2,4,4-trimethylhexan (UDMA).

Compositions of the invention that are free-radically polymerized preferably contain one or more suitable photopolymerization initiators that act as a source of free radicals when activated. Such initiators can be used alone or in combination with one or more accelerators and/or sensitizers. The photoinitiator should be capable of promoting free radical crosslinking of the ethylenically unsaturated moiety on exposure to light of a suitable wavelength and intensity. It also preferably is sufficiently shelf stable and free of undesirable coloration to permit its storage and use under typical dental conditions. Visible light photoinitiators are preferred. The photoinitiator frequently can be used alone, but typically it is used in combination with a suitable donor compound or a suitable accelerator (for example, amines, peroxides, phosphorus compounds, ketones and alpha-diketoine compounds).

Preferred visible light-induced initiators include camphorquinone (which typically is combined with a suitable hydrogen donor such as an amine), diaryliodonium simple or metal complex salts, chromophore-substituted halomethyl-s-triazines and halomethyl oxadiazoles. Particularly preferred visible light-induced photoinitiators include combinations of an alpha-diketone, e.g., camphorquinone, and a diaryliodonium salt, e.g., diphenyliodonium chloride, bromide, iodide or hexafluorophosphate, with or without additional hydrogen donors (such as sodium benzene sulfinate, amines and amine alcohols). Preferred ultraviolet light-induced polymerization initiators include ketones such as benzyl and benzoin, and acyloins and acyloin ethers. Preferred commercially available ultraviolet light-induced polymerization initiators include 2,2-dimethoxy-2-phenylacetophenone (“IRGACURE 651”) and benzoin methyl ether (2-methoxy-2-phenylacetophenone), both from Ciba-Geigy Corp.

The photoinitiator should be present in an amount sufficient to provide the desired rate of photopolymerization. This amount will be dependent in part on the light source, the thickness of the layer to be exposed to radiant energy, and the extinction coefficient of the photoinitiator. Typically, the photoinitiator components will be present at a total weight of about 0.01 to about 5%, more preferably from about 0.1 to about 5%, based on the total weight of the composition.

The compositions of the present invention may alternatively incorporate a mode of initiation of the polymerization reaction to initiate a crosslinking reaction without the need to expose the system to visible light. A preferred alternative mode for initiation of the polymerization reaction is the incorporation of an oxidizing agent and a reducing agent as a redox catalyst system to enable the dental composition to cure via a redox reaction.

The oxidizing agent should react with or otherwise cooperate with the reducing agent to produce free radicals capable of initiating polymerization of the ethylenically unsaturated moiety. The oxidizing agent and the reducing agent preferably are sufficiently shelf stable and free of undesirable coloration to permit their storage and use under typical dental conditions. The oxidizing agent and the reducing agent should also preferably be sufficiently soluble and present in an amount sufficient to permit an adequate free radical reaction rate. This can be evaluated by combining the ethylenically unsaturated moiety, the oxidizing agent and the reducing agent and observing whether or not a hardened mass is obtained.

Suitable oxidizing agents include persulfates such as sodium, potassium, ammonium and alkyl ammonium persulfates, benzoyl peroxide, hydroperoxides such as cumene hydroperoxide, tert-butyl hydroperoxide, tert-amyl hydroperoxide and 2,5-dihydroperoxy-2,5-dimethylhexane, salts of cobalt (III) and iron (III), hydroxylamine, perboric acid and its salts, salts of a permanganate anion, and combinations thereof. Hydrogen peroxide can also be used, although it may, in some instances, interfere with the photoinitiator, if one is present.

Preferred reducing agents include amines (and preferably aromatic amines), ascorbic acid, metal complexed ascorbic acid, cobalt (II) chloride, ferrous chloride, ferrous sulfate, hydrazine, hydroxylamine, oxalic acid, thiourea and salts of a dithionite, thiosulfate, benzene sulfinate, or sulfite anion.

Preferably used are such redox initiators as benzoyl peroxide/dimethyl aniline, cumene hydroperoxide/dimethyl aniline, cumene hydroperoxide/thiourea, ascorbic acid/Cu.sup.2+ salt, organic sulfinic acid (or salts thereof)/amine/peroxide; tributylborane, organic sulfinic acids and the like.

When redox initiator systems are used as photoinitiator systems, care must be taken to keep the reducing agent from reacting with the oxidizing agent before polymerization is desired. Generally, the use of a redox system necessitates providing the material in a two-part format.

For compositions that are polymerized by a cationic mechanism, suitable initiators include salts that are capable of generating cations such as the diaryliodonium, triarylsulfonium and aryldiazonium salts. Use of electronic donors or peroxides in such systems are also useful for enhancing rate of cure and depth of cure. Simultaneous photoinitiation of cationic and free radical groups may be afforded by, for example, onium salts or organometallic compounds in combination with or without oxidizing agents. Organometallic compounds can be selected from compounds that undergo sigma bond cleavage upon photolysis. The sigma bond is usually a metal-metal bond. Examples of suitable organometallic compounds include [CoFe(Co)₂]₂, Mn(CO)₆, Mn₂(CO)₁₀, in combination with iodonium salts and peroxides.

Fillers may be selected from one or more of any material suitable for incorporation in compositions used for medical applications, such as fillers currently used in dental restorative compositions and the like. As a rule, the filler is finely divided and preferably has a maximum particle diameter less than about 10 micrometers and an average particle diameter less than about 3.0 micrometers. More preferably, the filler has a maximum particle diameter less than about 2.0 micrometers and an average particle size of diameter less than about 0.6 micrometer. The filler can have a unimodal or polymodal (e.g., bimodal) particle size distribution. The filler can be an inorganic material. It can also be a crosslinked organic material that is insoluble in the polymerizable resin, and is optionally filled with inorganic filler. The filler should in any event be non-toxic and suitable for use in the mouth. The filler can be radiopaque, radiolucent or nonradiopaque.

Examples of suitable inorganic fillers are naturally-occurring or synthetic materials such as quartz, nitrides (e.g., silicon nitride), glasses derived from, for example Ce, Sb, Sn, Zr, Sr, Ba and Al, colloidal silica, feldspar, borosilicate glass, kaolin, talc, titania, and zinc glass; and sub-micron silica particles (e.g., pyrogenic silicas such as the “Aerosil” Series “OX 50”, “130”, “150” and “200” silicas sold by Degussa/Evonik and “Cab-O-Sil M5” silica sold by Cabot Corp.). Examples of suitable organic filler particles include filled or unfilled pulverized polycarbonates, polyepoxides, and the like. Preferred non-acid reactive filler particles are quartz, submicron silica. Mixtures of these non-acid reactive fillers are also contemplated, as well as combination fillers made from organic and inorganic materials such as pearl polymer fillers.

Preferably the surface of inorganic filler particles is treated with a coupling agent in order to enhance the bond between the filler and the polymerizable resin. The use of suitable coupling agents include gamma-methacryloxypropyltrimethoxysilane, gamma-mercaptopropyltriethoxysilane, gamma-aminopropyltrimethoxysilane, and the like.

Fillers may also be selected from fluoride releasing Materials. Fluoride releasing glasses, in addition provide the benefit of long-term release of fluoride in use, for example in the oral cavity. Fluoroaluminosilicate glasses are particularly preferred. Suitable acid reactive fillers are also available from a variety of commercial sources familiar to those skilled in the art. For example, suitable fillers can be obtained from a number of commercially available glass ionomer cements, such as “GC Fuji LC” and “Kerr XR” ionomer cement. Mixtures of fillers can be used if desired.

### Advantages of the Compositions According to the Invention

A The high reactivity of an acrylic acid ester containing urethane groups was combined with the rigid structure of the TCD skeleton and can thus be used as an alternative to bis-GMA in dental composites. In this way, higher degrees on conversion are achieved with an increased reactivity and contrary to the known connection between the degree of conversion and the volume shrinkage, low shrinkage composites are nevertheless made possible. Unexpectedly advantageous results are obtained in this case from the toxicological test with a proportion of acrylate resin of >5% (Examples/Attachments).
B The toxicological tests show the surprisingly high biocompatibility of the polymerised composite.
C Higher degrees of polymerisation are advantageous for the mechanical properties of the composites, although acrylate monomers were considered to be unsuitable crosslinking agents as a result of the disadvantageous toxicological property. After curing, a highly favourable biocompatibility has surprisingly been detected.
   The dental composites are used in direct and indirect odontology.

The following Examples are intended to explain the invention without limiting it. As far as parts or percentages are given these are—as well as in the remaining specification—based on weight unless otherwise indicated.

### EXAMPLE

### Composite Paste (According to the Invention)

The formulation was effected in the kneader with a planetary gear. The work needs to be carried out under yellow light.

Monomers, initiators and auxiliary agents are provided (possibly already pre-dissolved) and homogenised with 2500 RPM for 10 min.

The filler is weighed and added in several portions of decreasing quantity ([%]: 35/25/20/10/5/5). Following each addition, homogenising is again carried out until a kneadable paste has formed. If the paste warms up strongly before the next mixing operation, it should be cooled slightly. If filler residues remain, the mixing process is repeated once more.

### Toxicological Testing (Cytotoxicity Testing In Vitro by Forming an XXT Dye)

Using the XTT dye test, the ability to divide and the survival rate of the cells are evaluated simultaneously via a colorimetric determination. The test is based on the liberation of the yellow tetrazolium salt XTT (sodium-3′-(1-phenylaminocarbonyl)-3,4-tetrazolium)bis(4-methoxy-6-nitro)benzene sulphonic acid hydrate), which forms an orange-coloured water-soluble formazan dye as a result of the dehydrogenase activity of active mitochondria.

The test of the cytotoxicity took place according to the standard requirements according to ISO 10993-5 and DIN EN ISO 7405. For this purpose, the non-sterile material specimen was extracted with stirring for 72±2 hours at 37±1° C. (extraction agent: Dubecco's modified eagle medium (DMEM), 10% fetal calf serum (FCS) was added). The ratio of surface/volume was 6 cm²/ml. Subsequently, the extract was filtered aseptically.

A positive and a negative control regarding the cell culture passed through the test in parallel as a reference for validation. The negative control was extracted with a ratio of weight/volume of 1 g/5 ml medium. The positive control was extracted with a ratio of weight/volume of 6 cm²/ml of the culture medium (DMEM 10% FCS) for 72±2 hours at 37±1° C.

Negative control: polyethylen (Greiner Cellstart, item. No. 188271, batch no. 04080197).

Positive control: powder-free industrial latex gloves (Semperit GmbH, batch no. 67910077).

The test was carried out with L929 cells (ATCC No. CCL1, NCTC clone 929 (connective tissue mouse), clone of strain L (DSMZ)). For the test, cultures in 75 cm² culture flasks (Greiner) in DMEM (PAA) with 10% FCS (Seromed) were used at 37±1° C. and 5.0% carbon dioxide.

The cell cultures were treated with PBS free from Ca—Mg for approximately 3 minutes. The enzymatic reaction is stopped with DMEM 10% FCS and a single cell suspension with a concentration of 2·10⁴ cells/ml is produced. 100 μl of this suspension are introduced into the cavities of a microtitre plate. The cell culture was incubated for 24±2 hours at 37±1° C. using 5.0% CO₂ and 95% air.

Subsequently, dilutions of the extract with DMEM 10% FCS to concentrations of 100, 80, 50, 30, 20, 10% by vol. were provided in a further microtitre plate. Then, the cell culture medium of the previously prepared cells is removed and 100 μl of the dilutions of the test extract are mixed with 100 μl of the control (100% concentration) in 3 samples respectively. The cultures are incubated for 24±2 hours at 37±1° C. using 5.0% CO₂ and 95% air.

The XTT dye begins 1-2 hours before the end of the incubation period. For this purpose, 50 μl of the XTT dye mixture (Roche Diagnostics) are added to each cell culture. The mixture consists of XTT marker reagent (5 ml) and the electron coupling reagent (0.1 ml). On completion of the incubation period (1-2 hours), the cell cultures are introduced into a plate detector (Biotek Systems) for calorimetric analysis. During this process, the absorption is recorded at 490 nm and evaluated in comparison with the reference wavelength of 630 nm.

A reduction in the number of living cells corresponds to a decrease in the activity of the dehydrogenase of the mitochondria in the cell cultures concerned. As a result, the formation of the orange-coloured formazan dye is reduced in direct correlation and recorded quantitatively as extinction.

$$Activity of the mitochondria dehydrogenase [ \% ] = \frac{\begin{matrix}A ( sample , 490 nm ) - \\ A ( reference , 490 nm )\end{matrix}}{\begin{matrix}A ( control , 490 nm ) - \\ A ( reference , 490 nm )\end{matrix}}$$

$$\begin{matrix}A ( sample , 490 nm ) & absorption with test extract \\ A ( reference , 490 nm ) & absorption of the empty medium ( without cells ) \\ A ( control , 490 nm ) & absorption with control culture without extract\end{matrix}$$

The result was determined as the arithmetic mean with the standard deviation for a set of three samples respectively. The dehydrogenase activity of less than 70% is assessed as being clearly cytotoxic.

### Discussion of the Results

The stronger cytotoxicity of acrylates in comparison with methacrylate monomers with a comparable molecular weight, polarity and degree of functionalisation is well known. For this reason, pure mixtures of different methacrylates or only small proportions of acrylate monomers are preferably used in dental materials.

In agreement with this known fact, the author's own investigations with proportions of different acrylate monomers (Sartomer 368 and 295) also showed a detectably higher cytotoxicity vis-à-vis a comparable preparation from methacrylates without these additions. Whereas the test composite 201 in paste form of a common composition of a dental resin of bis-GMA and triethylene glycol dimethacrylate (TEGDMA) corresponds to a ratio 7:3 and exhibits no cytotoxic potential, a clear increase in the cytotoxicity can be observed in the case of sample 204 with an addition of multifunctional acrylate monomers.

In contrast to this known effect, a comparable composite exhibits in fact a reduction of the cytotoxic effectiveness when bis-GMA is exchanged for the diacrylate-functional TCD monomer. The TCD monomer according to the invention reduces demonstrably the cytotoxic potential in conventional dental composite materials.

The tests were reproduced in another resin mixture with urethane methacrylate. A mixture of triethylene glycol dimethacrylate, UDMA and the TCD monomer was tested in different combinations with further monomers. In order to achieve a comparability with the conventional bis-GMA/TEGDMA composite, 72% bis-GMA was added in one test and the sample 338 was tested. Using the hardened composite, a very low cytotoxic potential was detected which was below the effectiveness of sample 230. The complete replacement of bis-GMA by the comparable low-shrinkage acrylate monomer TCD-DI-HEA led to a similarly advantageous cytotoxic potential in the samples 349 and 350, it being possible to reduce the initiator content even further as a result of the higher reactivity of the monomer.

On the other hand, variations of this mixture with approximately 10-15% multifunctional acrylate monomers (SR295) exhibited a clearly cytotoxic effectiveness of the polymerised composite samples.

In this way, the same connection between the cytotoxic effectiveness and the type of acrylate monomers contained could be shown also for a differently composed resin mixture. In the tests carried out, it was possible to show that the acrylate monomer according to the invention with a TCD-urethane structure results in more advantageous toxicological properties than the usually used, more reaction-inert methacrylate monomers or other reactive acrylate monomers.

The very low cytotoxic potential of hardenable dental materials with the monomer TCD-DI-HEA according to the invention which represent a medical product and remain usually in constant contact with the living tissue is of central importance for the usability and biological acceptance of such materials by patients and users.

TABLE I
Results of the cytotoxicity measurements

| | | | | | | | | Mitochondrial hydrogenase activity | | | | | | |
|---|---|---|---|---|---|---|---|---|---|---|---|---|---|---|
| | | | | | | | | in the case of | | | | | | |
| | | Bis | | | SR 295 | | SR 368 | extract concentrations in % | | | | | | |
| Type | TCD | GMA | TEDMA | UDMA | Tetra A | UTMA | Tri A | 100 | 80 | 50 | 30 | 20 | 10 | Evaluation |
| completely poly | | | | | | | | | | | | | | |
| completely poly | | | | | | | | | | | | | | |
| Dye chips | | | | | | | | | | | | | | |
| Sample 5 | | 68% | 32% | | | | | 88 | 91 | 94 | 97 | 98 | 96 | no cytotoxic potential |
| 201 | | | | | | | | | | | | | | |
| Sample 1 | | 38% | 13% | 19% | 10% | | 20% | 0 | 1 | 18 | 71 | 90 | 98 | marked cytotoxic potential |
| 204 | | | | | | | | | | | | | | |
| Sample 6 | 80% | | 20% | | | | | 86 | 94 | 96 | 99 | 99 | 96 | no cytotoxic potential |
| 230 | | | | | | | | | | | | | | |
| Sample 4 | 60% | | 25% | | 15% | | | 46 | 74 | 91 | 95 | 97 | 99 | marked cytotoxic potential |
| 332 | | | | | | | | | | | | | | |
| Sample 2 | 60% | | 16% | 12% | 12% | | | 10 | 40 | 85 | 95 | 97 | 98 | marked cytotoxic potential |
| 337 | | | | | | | | | | | | | | |
| Sample 3 | 54% | | 17% | 13% | 13% | 3% | | 12 | 52 | 88 | 93 | 96 | 100 | marked cytotoxic potential |
| hardened | | | | | | | | | | | | | | |
| Sample 9 | 13% | 73% | 4% | 6% | | 4% | | 94 | 94 | 97 | 98 | 99 | 98 | no cytotoxic potential |
| polymer 338 | | | | | | | | | | | | | | |
| Sample 7 | 90% | | 2% | 4% | | 4% | | 89 | 92 | 96 | 98 | 100 | 99 | no cytotoxic potential |
| Polymer 349 | | | | | | | | | | | | | | |
| Sample 8 | 90% | | 2% | 4% | | 4% | | 91 | 93 | 98 | 100 | 100 | 99 | no cytotoxic potential |
| polymer 350 | | | | | | | | | | | | | | |

1. Dental composites comprising
monomers,
crosslinking agents,
fillers,
initiators, wherein
A: the proportion of crosslinking agent is formed in an amount of more than 50% by acrylate monomers with a TCD urethane structure having the general formula 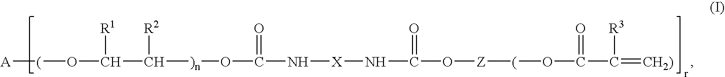
in which
A is a straight-chain or branched aliphatic radical with 2 to 20 carbon atoms, the radical containing, optionally, 1 to 3 oxygen bridges, an aromatic radical with 6 to 24 carbon atoms, an araliphatic radical with 7 to 26 carbon atoms or a cycloaliphatic radical with 6 to 26 carbon atoms,
r represents the number of chains issuing from A and an integer of 2 to 6,
R¹ and R² are identical and represent hydrogen or are different and represent hydrogen and methyl,
n represents, for each chain issuing from A, independently an integer of 0 to 5,
X represents the group 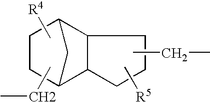
in which
R⁴ and R⁵ are identical or different and represent hydrogen, halogen, lower alkoxy, lower alkyl or trifluoromethyl,
Z may contain a divalent straight-chain or branched aliphatic hydrocarbon radicals of 3 to 15 carbon atoms which, optionally, may contain 1 to 3 oxygen bridges atoms and, optionally, may be substituted by 1 to 4 additional (meth)acrylate radicals, R², R³ are exclusively hydrogen, and
B exhibits the cytotoxicity of the hardened composite corresponding to the standard requirements according to ISO 10993-5 and DIN EN ISO 7405, has the assessment “no cytotoxic potential”.

2. Composition according to claim 1, wherein the crosslinking agent moiety further comprises silorans.

3. Composition according to claim 1 which is essentially free from bis-GMA.
